(19) United States
(12) Patent Application Publication
(10) Pub. No.: US 2004/0152148 A1
(43) Pub. Date: Aug. 5, 2004

(54) Novel methods involving the determination of activity of enzymes that use or produce prostaglandin endoperoxide H2

(75) Inventor: Ralph H. Lambalot, Wrentham, MA (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/763,860

(22) Filed: Jan. 23, 2004

Related U.S. Application Data
(60) Provisional application No. 60/442,876, filed on Jan. 27, 2003.

(51) Int. Cl. C12Q 1/26

(52) U.S. Cl. 435/25

(57) ABSTRACT

The present invention provides assays for determining the activity of an enzyme that uses or produces prostaglandin endoperoxide H₂ (PGH₂), methods of using the assays to screen for potential modulators of such enzymes, and kits for practice of the assays.

This application claims priority, under 35 U.S.C. §119(e), from U.S. provisional patent application Serial No. 60/442,876, which was filed Jan. 27, 2003, the disclosure of which is hereby incorporated by reference.

### FIELD OF THE INVENTION

The present invention relates to methods of determining the activity of enzymes that use or produce prostaglandin endoperoxide H₂ (PGH₂) such as, for example, prostaglandin E synthase (PGES). The invention also provides methods for identifying and testing modulators of enzymes that use or produce PGH₂, as well as kits for the practice of the same.

### BACKGROUND OF THE INVENTION

Prostaglandins (which include PGH₂, PGE₂, PGD₂, PGF₂α, PGI₂ and other related compounds) represent a diverse group of autocrine and paracrine hormones that are derived from the metabolism of fatty acids. They belong to a family of naturally occurring eicosanoids (prostaglandins, thromboxanes and leukotrienes) which are not stored as such in cells, but are biosynthesized on demand from arachidonic acid, a 20-carbon fatty acid that is derived from the breakdown of cell-membrane phospholipids. Under normal circumstances, the eicosanoids are produced at low levels to serve as important mediators of many and diverse cellular functions which can vary considerably in different types of cells. Prostaglandins also play critical roles in pathophysiology. In particular, inflammation is both initiated and maintained, at least in part, by the overproduction of prostaglandins in injured cells. The central role that prostaglandins play in inflammation is underscored by the fact that those aspirin-like non-steroidal anti-inflammatory drugs (NSAIDS) that are most effective in the therapy of many pathological inflammatory states all act by inhibiting prostaglandin synthesis.

Prostaglandin endoperoxide H₂ (PGH₂) is formed from arachidonic acid by the action of cyclooxygenases (COX)-1 or -2. COX-1 is constitutively expressed in many cells and tissues, whereas the COX-2 protein can be induced by proinflammatory cytokines such as interleukin-1β at sites of inflammation. Downstream of the cyclooxygenases, the product PGH₂ is further metabolized into various physiologically important eicosanoids, e.g., PGF_{2α}, PGE₂, PGD₂, PGI₂ (prostacyclin) and thromboxane (TX) A₂ (Smith, W. L., Am. J. Physiol., 263, F181, 1992) by a variety of enzymes. Prostaglandin E synthase is an enzyme that catalyzes the conversion of a cyclic endoperoxide substrate into a 9-keto, 11α hydroxy form of the substrate. PGES catalyzes, e.g., the conversion of precursor molecules into PGE₂ and analogs thereof, e.g., synthetic analogs. For example, PGES catalyzes the conversion of PGH₂ into PGE₂.

### SUMMARY OF THE INVENTION

The present invention provides, in part, methods for determining the activities of enzymes that use or produce PGH₂, but do not produce malondialdehyde. In a first aspect, methods for determining in a sample the activities of enzymes that use PGH₂ may comprise: (a) contacting a sample with a reducing agent under conditions suitable to substantially convert PGH₂ into malondialdehyde and thereby obtain a reacted sample; (b) contacting the reacted sample with a malondialdehyde detection reagent under conditions suitable to substantially convert the malondialdehyde into a detectable compound; and (c) determining the amount of the detectable compound, wherein the activity of the enzyme in the sample is inversely proportional to the amount of the detectable compound. In one embodiment of the first aspect, the enzyme may be selected from the group consisting of prostaglandin synthases and prostacyclin synthases, and in one such embodiment is a prostaglandin synthase. In one embodiment, the prostaglandin synthase is prostaglandin E synthase (PGES). In one embodiment of the invention, the reducing agent is ferrous chloride. In other embodiments of the invention the detection agent may be 2-thiobarbituric acid (TBA) or a 2-thiobarbituric acid derivative. In one embodiment of the invention, the detectable compound is a fluorescent compound.

In a second aspect, methods for determining in a sample the activities of enzymes that produce PGH₂ may comprise: (a) contacting a sample with a reducing agent under conditions suitable to substantially convert PGH₂ into malondialdehyde and thereby obtain a reacted sample; (b) contacting the reacted sample with a malondialdehyde detection reagent under conditions suitable to substantially convert the malondialdehyde into a detectable compound; and (c) determining the amount of the detectable compound, wherein the activity of the enzyme in the sample is proportional to the amount of the detectable compound. In one such embodiment, the enzyme is a PGH₂ synthase.

The present invention also provides methods of identifying and testing modulators of enzymes that use or produce PGH₂ but do not produce malondialdehyde. In one aspect, methods of identifying and testing modulators of enzymes that use or produce prostaglandins may comprise: (a) contacting a sample containing an enzyme with a test compound; (b) contacting the sample with a reducing agent under conditions appropriate to convert PGH₂ into malondialdehyde and thereby obtain a reacted sample; (c) contacting the reacted sample with a malondialdehyde detection reagent under conditions appropriate to convert the malondialdehyde into a detectable compound; and (d) determining the amount of the detectable compound., wherein the amount of detectable compound is used to determine whether or not the test compound modulates the activity of the enzyme. In some embodiments, the amount of detectable compound is determined in a sample at multiple time points, wherein a change in the amount of detectable compound over time indicates that the test compound modulates the activity of the enzyme. In other embodiments, the amount of detectable compound is determined in the sample contacted with a test compound, and compared to the amount of detectable compound determined in a sample containing the enzyme wherein the enzyme has not been contacted with the test compound. In some embodiments, the enzyme may be purified or isolated. In one embodiment, the enzyme may be partially purified. In some embodiments, the test compound may be a small molecule.

The invention further provides kits for determining the activities of enzymes that use or produce PGH₂ but do not produce malondialdehyde as a product including a reducing agent and/or a malondialdehyde detection reagent, and optionally instructions for their use.

Other features and advantages of the invention will be apparent from the following detailed description, and from the appendant claims.

### BRIEF DESCRIPTION OF THE FIGURES

FIG. 1 depicts the chemical reactions occurring in the three steps of one exemplary embodiment of the assay.

FIG. 2 depicts a titration of PGH₂ using an exemplary embodiment of the assay, wherein the raw fluorescence reading is plotted versus the concentration of PGH₂.

FIG. 3 depicts the percent conversion of PGH₂ by PGES over time, as measured by an exemplary embodiment of the assay.

FIG. 4 depicts the percent conversion of PGH₂ by varying PGES, as measured by an exemplary embodiment of the assay.

FIG. 5 depicts at left the structure of MK886, a commercially available inhibitor of PGES. At right are depicted the results of the use of one embodiment of the assay to measure the ability of MK886 to inhibit PGES. The IC50 of MK886, as measured by the subject assay, is depicted at the top of the figure.

### DETAILED DESCRIPTION OF THE INVENTION

A. General

Assays have been developed for determining the activities of enzymes that use or produce prostaglandins, but do not produce malondialdehyde as a by-product. In general, a reducing agent is first employed to convert unreacted prostaglandin to malondialdehyde. The malondialdehyde is then reacted with a malondialdehyde detection reagent to produce a detectable adduct. Human prostglandin E2 synthase mediates the conversion of PGH₂ to PGE₂. Ferrous chloride has been utilized to convert unreacted PGH₂ into malondialdehyde (MDA) and 12-hydroxyheptadecatrienoic acid (12HHT). The malondialdehyde was then reacted with thiobarbituric acid to form a fluorescent adduct that was quantitated directly using fluorometry. The amount of fluorescent adduct produced correlated with the amount of PGH₂ present in the sample, and hence may be used to monitor the conversion of PGH₂ by the PGES enzyme.

B. Definitions

For convenience, before further description of the present invention, certain terms employed in the specification, examples, and appendant claims are collected here. These definitions should be read in light of the entire disclosure and understood as by a person of skill in the art.

The singular forms “a”, “an”, and “the” include plural references unless the context clearly dictates otherwise.

“Biological activity” or “bioactivity” or “activity” or “biological function”, which are used interchangeably, for the purposes herein means the function performed by an enzyme that uses or produces PGH₂, e.g., one with the ability to catalyze the formation of PGE₂ from PGH₂.

“Comprise” and “comprising” are used in the inclusive, open sense, meaning that additional elements may be included.

A “combinatorial library” or “library” is a plurality of compounds, which may be termed “members,” synthesized or otherwise prepared from one or more starting materials by employing either the same or different reactants or reaction conditions at each reaction in the library. In general, the members of any library show at least some structural diversity, which often results in chemical diversity. A library may have anywhere from two different members to about 10⁸ members or more. In certain embodiments, libraries of the present invention have more than about 12, 50 and 90 members. In certain embodiments of the present invention, the starting materials and certain of the reactants are the same, and chemical diversity in such libraries is achieved by varying at least one of the reactants or reaction conditions during the preparation of the library. Combinatorial libraries of the present invention may be prepared in solution or on the solid phase.

An “enzyme that uses PGH₂, but does not produce malondialdehyde” refers to any enzyme for which a PGH₂ is a substrate, wherein the reaction of the enzyme with PGH₂ does not result in a malondialdehyde being a product of the reaction. Such an enzyme may be, for example, an isomerase that rearranges PGH₂ into another structure, a reductase that reduces PGH₂ to form another molecule, or a synthase which converts PGH₂ into a new molecule. Non-limiting examples of enzymes that use PGH₂ include prostaglandin synthases and prostacylcin synthases. Examples of prostaglandin synthases which catalyze conversion of PGH₂ into other prostglandins include, but are not limited to, prostaglandin E synthases (PGES), prostaglandin D synthases (PGDS), and prostaglandin F synthases (PGFS). An example of a prostacyclin synthase which catalyzes interconversion of or otherwise uses PGH₂ in its catalytic action includes, but is not limited to, prostaglandin I2 synthase (PGIS).

An “enzyme that produces PGH₂, but does not produce malondialdehyde” refers to any enzyme which synthesizes PGH₂, wherein the reaction of the enzyme to form PGH₂ does not result in a malondialdehyde as a product of the reaction. Non-limiting examples of enzymes that produce PGH₂ include PGH₂ synthases.

“Fragment”, when used in reference to a reference polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

“Including” is used herein to mean “including but not limited to”. “Including” and “including but not limited to” are used interchangeably.

“Interact” is meant to include detectable interactions between molecules, such as may be detected using, for example, a hybridization assay. Interact also includes “binding” interactions between molecules. Interactions may be, for example, protein-protein, protein-nucleic acid, protein-small molecule or small molecule-nucleic acid in nature.

“Isolated polypeptide” refers to a polypeptide, which may be prepared from recombinant DNA or RNA, or be of synthetic origin, some combination thereof, may be a fraction from a microscomal preparation, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

“Label” or “labeled” refer to incorporation or attachment, either covalently or non-covalently, of a detectable marker into a molecule, such as a malondialdehyde detection reagent. Any suitable method of labeling molecules may be used with the invention. Examples of labels include, but are not limited to radioisotopes, fluorescent labels, heavy atoms, chemiluminescent groups, and/or biotinyl groups.

“Malondialdehyde detection reagent” refers to any molecule or compound that may be used to detect the presence of malondialdehyde in a sample. Such a molecule or compound may form a spectroscopically or otherwise detectable covalent adduct with malondialdehyde, may precipitate malondialdehyde out of solution, and/or may convert malondialdehyde into another molecule.

“Modulation”, when used in reference to a functional property or biological activity or process (e.g., enzyme activity or receptor binding), refers to the capacity to either upregulate (e.g., activate or stimulate), downregulate (e.g., inhibit or suppress) or otherwise change a quality of such property, activity or process. In certain instances, such regulation may be contingent on the occurrence of an event, such as activation of a signal transduction pathway, and/or may be manifest only in particular cell types.

“Modulator” refers to a polypeptide, nucleic acid, macromolecule, complex, molecule, e.g., a small molecule of molecular weight less than 1000 daltons, compound, species or the like (naturally-occurring or non-naturally-occurring), or a large molecule of molecular weight over 1000 daltons, or an extract made from biological materials such as bacteria, plants, fungi, or animal cells or tissues, that is capable of causing modulation. Modulators can be evaluated for potential activity as inhibitors or activators (directly or indirectly) of a functional property, biological activity or process, or combination of them (e.g., agonist, partial antagonist, partial agonist, inverse agonist, antagonist, anti-microbial agents, inhibitors of microbial infection or proliferation, and the like) by inclusion in assays. In such assays, an individual modulator or any suitable combination of modulators may be screened. The activity of a given modulator may be known, unknown or partially known. A modulator may be adaptable for use as a pharmaceutical.

“PGES” or “PGE synthase” is prostagladin E synthase, an enzyme that catalyzes the conversion of a cyclic endoperoxide substrate into a 9-keto, 11α hydroxy form of the substrate. PGES catalyzes, e.g., the conversion of precursor molecules into PGE₂ and analogs thereof, e.g., synthetic analogs. For example, PGES catalyzes the conversion of PGH₂ into PGE₂.

“PGH₂” is prostaglandin endoperoxide H₂.

“Purified” refers to an object species that is the predominant species present (i.e., on a molar basis it is more abundant than any other individual species in the composition). In a “partially purified” composition the object species comprises at least about 50 percent (on a molar basis) of all species present. In making the determination of the purity of a species in solution or dispersion, the solvent or matrix in which the species is dissolved or dispersed need not be included in such determination; instead, only the species (including the one of interest) dissolved or dispersed are taken into account. Generally, a “purified” composition will have one species that comprises more than about 85 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. Purification of a protein may be accomplished using standard techniques for protein purification in light of the teachings herein. Purity of a polypeptide may be determined by a number of methods known to those of skill in the art, including, for example, amino-terminal amino acid sequence analysis, gel electrophoresis and mass-spectrometry analysis.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid. Such recombinant polypeptides may comprise, and optionally be purified from, e.g., whole cell lysates or microsomal preparations. Further, such recombinant polypeptides may comprise stabilized polypeptide preparations derived from microsomal preparations.

A “reducing agent” refers to any molecule or compound that donates electron(s) in a chemical reaction and becomes oxidized when another substance is reduced.

A “sample” includes material obtained from a subject. For example, samples may be obtained from a human or animal subject, a plant, a cell culture or an environmental location, such as a water or an air sample. Sample also includes materials that have been processed or mixed with other materials. For example, a blood sample may be processed to obtain serum, red blood cells, etc., each of which may be considered a sample.

“Small molecule” refers to a composition that has a molecular weight of less than about 1000 daltons. Small molecules may be nucleic acids, peptides, polypeptides, peptidomimetics, carbohydrates, lipids or other organic (carbon-containing) or inorganic molecules. As those skilled in the art will appreciate, based on the present description, libraries of chemical and/or biological extensive libraries of chemical and/or biological mixtures, and fungal, bacterial, or algal extracts, may be screened with any of the assays of the invention to identify compounds that are modulators.

“Test compound” refers to a molecule to be tested by one or more screening method(s) as a putative modulator of an enzyme that uses or produces PGH₂. A test compound is usually not known to bind to a target of interest. The term “control test compound” refers to a compound known to bind to the target (e.g., a known agonist, antagonist, partial agonist or inverse agonist). The term “test compound” does not include a chemical added as a control condition that alters the function of the target to determine signal specificity in an assay. Such control chemicals or conditions include chemicals that 1) nonspecifically or substantially disrupt protein structure (e.g., denaturing agents (e.g., urea or guanidinium), chaotropic agents, sulfhydryl reagents (e.g., dithiothreitol and β-mercaptoethanol), and proteases), 2) generally inhibit cell metabolism (e.g., mitochondrial uncouplers) and 3) non-specifically disrupt electrostatic or hydrophobic interactions of a protein (e.g., high salt concentrations, or detergents at concentrations sufficient to non-specifically disrupt hydrophobic interactions). Examples of test compounds include, but are not limited to, peptides, nucleic acids, carbohydrates, biologically inactive substrates, and small molecules. The term “novel test compound” refers to a test compound that is not in existence as of the filing date of this application. In certain assays using novel test compounds, the novel test compounds comprise at least about 50%, 75%, 85%, 90%, 95% or more of the test compounds used in the assay or in any particular trial of the assay.

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). A variant may have “nonconservative” changes, wherein a substituted amino acid has different structural or chemical properties (e.g., replacement of glycine with tryptophan). Analogous minor variations may also include amino acid deletions or insertions, or both. Those skilled in the art will appreciate that guidance in evaluating which amino acid residues may be substituted, inserted, or deleted without abolishing biological or immunological activity may be found using computer programs well known in the art, for example, LASERGENE software (DNASTAR).

C. Detectable Enzyme Activities

The activity of any enzyme that uses or produces PGH₂ via a reaction may be evaluated using the subject assays. Enzymes that produce PGH₂ include PGH₂ synthases such as the cyclooxygenases (COX)-1 or -2. Enzymes that use or catalyze specific interconversions of PGH₂ include prostaglandin synthases, prostacyclin synthases, and thromboxane synthases. The activity of prostaglandin synthases, prostacyclin synthases, and any other enzyme that uses or produces PGH₂, which do not produce a malondialdehyde side product may be detected using the subject assays. Examples of prostaglandin synthases which catalyze conversion of PGH₂ into other prostglandins include, but are not limited to, prostaglandin E synthases (PGES), prostaglandin D synthases (PGDS), prostaglandin F synthases (PGFS), and the like. Examples of prostacyclin synthases which catalyze interconversions of or otherwise use PGH₂ in their catalytic action include, but are not limited to, prostaglandin 12 synthase (PGIS) and the like.

In some embodiments, the enzyme is PGES. Purified preparations of PGES and variants thereof have been made and are the subject of pending application “Methods for Preparing Prostaglandin E Synthase”, U.S. Ser. No. 10/227,617, filed Aug. 23, 2002. Isolated PGE synthase and assays for modulators of PGE synthase activity are disclosed in U.S. Pat. No. 6,395,502 B1. Both U.S. Ser. No. 10/227,617 and U.S. Pat. No. 6,395,502 B1 are hereby incorporated by reference in their entireties. The activity of the enzymes and variants and analogs prepared as taught in this application may be assayed using the methods of the present invention.

Fragments of any of the above enzymes may also be used with the assays of the invention, as may analogs or variants of the enzymes. Enyzmes for use with the assays of the present invention may be naturally-occurring, e.g. purified from a native source, or recombinant.

D. Assays

Methods for determining the activities of enzymes that use or produce PGH₂ may comprise: (a) contacting a sample with a reducing agent under conditions appropriate to convert PGH₂ into malondialdehyde and thereby obtain a reacted sample; (b) contacting the reacted sample with a malondialdehyde detection reagent under conditions appropriate to convert the malondialdehyde into a detectable compound; and (c) determining the amount of the detectable compound.

D.1. Reducing Agents

The reducing agent used to convert PGH₂ to malondialdehyde in the subject assays may be selected from any of the gentler reducing agents recognized in the art. Generally, the reduction may be carried out under aqueous conditions at neutral pH and room temperature using anywhere from 2 to 20 equivalents of the reducing agent per mole of PGH₂. In one preferred embodiment, the reducing agent used in the assay is ferrous chloride (FeCl₂).

D.2. Malondialdehyde Detection Reagents

Any molecule or compound able to detect or make detectable the presence of malondialdehyde (MDA) in a sample may be used in the subject assays. Such agents may produce a labeled adduct, visible product, fluorescent product, or an isotopically-labeled product. In certain embodiments, e.g. for example when the level of PGH₂ to be detected may be below the limit of a colorimetric or fluorescence assay, the above-described detection reagents may be isotopically-labeled, e.g. with ³H, ¹⁴C, ³⁵S, ³³P, ³²P, and the like.

In one preferred embodiment, the malondialdehyde detection reagent is 2-thiobarbituric acid (TBA). The reaction between malondialdehyde (MDA) and 2-thiobarbituric acid (TBA) yields about a 1:2 adduct that is both pigmented and fluorescent. See Scheme 1, provided immediately below.

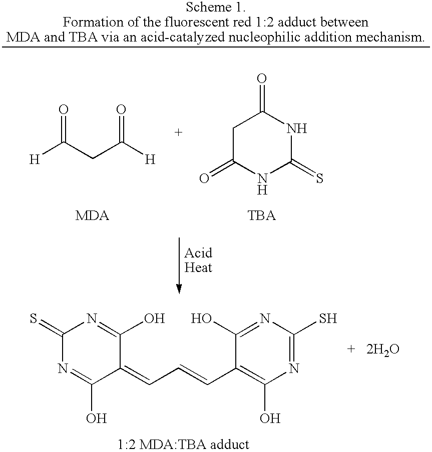

As those skilled in the art will appreciate, the detection of MDA through fluorescence of a 1:2 adduct need not be limited to adducts of TBA and its derivatives. Detectable fluorescent compounds are obtained when MDA forms a 1:2 adduct with a primary amine. See Scheme 2, provided immediately below.

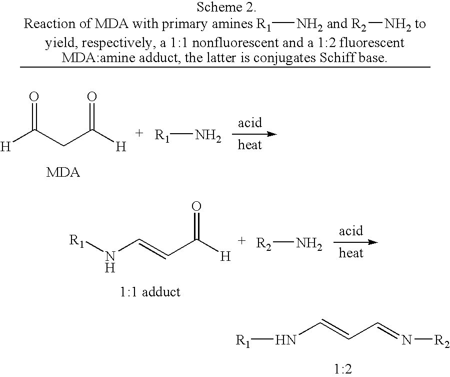

R₁ and R₂ independently can be any organic group commonly used in the formation of Schiff bases including but not limited to alkyl, alkenyl, alkynyl, aryl, heteroaryl, aralkyl, or alkaryl groups. Because formation of a Schiff base produces one equivalence of water, a means of removing water from the reaction is generally used to drive the reaction over to the right. Two common methods of water removal include heat and the use of a drying agent.

The reaction can also be carried out with derivatives of TBA where the nitrogens are substituted with, for example, alkyl, alkenyl, alkynyl, aryl, heteroaryl, aralkyl, or alkaryl groups. Two common but non-limiting examples include 1,3-diethyl-2-thiobarbituric acid and 1,3-diphenylbarbituric acid depicted below.

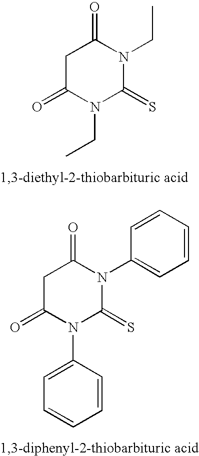

However, the malondialdehyde detection reagents are not limited to TBA and TBA-derivatives. Rather, as mentioned above, any molecule or compound able to detect or make detectable the presence of malondialdehyde may be used. In other embodiments, antibodies directed against malondialdehyde may be produced using well-known techniques in the art for eliciting specific antibodies and used as detection agents in the assays of the invention. In one embodiment, malondialdehyde may be immunoprecipitated from a sample. In another embodiment, an anti-MDA monoclonal antibody may be conjugated to a detectable label and reacted with a sample potentially containing MDA. In such embodiments, MDA may be detected via an enzyme-linked immunosorbent assay (ELISA) format. In other embodiments, an aldehyde reactive probe such as AMCA-hydrazide (Molecular Probes, Eugene, Oreg.) or any other fluorescent or otherwise labeled hydrazine derivatives including semicarbazides and carbohydrazides may be used to detect MDA in a sample.

E. Methods of Use of the Subject Assays

The present invention provides methods of using the above-described assays to detect the activity of an enzyme that uses or produces PGH₂. In one embodiment, methods for determining in a sample the activities of enzymes that use PGH₂ may comprise: (a) contacting a sample with a reducing agent under conditions suitable to substantially convert PGH₂ into malondialdehyde and thereby obtain a reacted sample; (b) contacting the reacted sample with a malondialdehyde detection reagent under conditions suitable to substantially convert the malondialdehyde into a detectable compound; and (c) determining the amount of the detectable compound, wherein the activity of the enzyme in the sample is inversely proportional to the amount of the detectable compound. In one embodiment, the enzyme may be selected from the group consisting of prostaglandin synthases and prostacyclin synthases, and in one embodiment is a prostaglandin synthase. In one embodiment, the prostaglandin synthase is prostaglandin E synthase (PGES). In one embodiment of the invention, the reducing agent is ferrous chloride. In other embodiments of the invention the detection agent may be 2-thiobarbituric acid (TBA) or a 2-thiobarbituric acid derivative. In one embodiment of the invention, the detectable compound is a fluorescent compound.

In other embodiments, methods for determining in a sample the activities of enzymes that produce PGH₂ may comprise: (a) contacting a sample with a reducing agent under conditions suitable to substantially convert PGH₂ into malondialdehyde and thereby obtain a reacted sample; (b) contacting the reacted sample with a malondialdehyde detection reagent under conditions suitable to substantially convert the malondialdehyde into a detectable compound; and (c) determining the amount of the detectable compound, wherein the activity of the enzyme in the sample is proportional to the amount of the detectable compound. In one embodiment, the enzyme is a PGH₂ synthase.

Such methods may be used, for example, in determining the activity of a pharmaceutical, cosmetic, reagent or other such commercial preparation comprising an enzyme that uses or produces PGH₂. Such methods may also be used in evaluting the activity of an enzyme that uses or produces PGH₂ during a purification protocol, or for determining the level of activity of such an enzyme, e.g. in a sample of purified protein, a sample taken from a subject, etc. Such methods could also be used to determine whether or not a fragment, analog, or variant of such enzymes retained biological activity.

In still other embodiments, the assays may be used to determine the level of enzyme activity in a subject, e.g. in a sample taken from such a subject. The sample may be at least partially or fully purified, if desired, before the assay is performed.

Assaying biological activity may be accomplished in any vessel suitable for containing the reactants. Examples include microtitre plates, test tubes, and micro-centrifuge tubes. Such assays may be cell-free. In certain embodiments, the enzyme is purified, or partially purified.

The present invention further provides methods of screening modulators of the activity of an enzyme that uses or produces PGH₂. In one embodiment, methods of identifying and testing modulators of enzymes that use or produce prostaglandins may comprise: (a) contacting a sample containing an enzyme with a test compound; (b) contacting the sample with a reducing agent under conditions appropriate to convert PGH₂ into malondialdehyde and thereby obtain a reacted sample; (c) contacting the reacted sample with a malondialdehyde detection reagent under conditions appropriate to convert the malondialdehyde into a detectable compound; and (d) determining the amount of the detectable compound., wherein the amount of detectable compound is used to determine whether or not the test compound modulates the activity of the enzyme. In some embodiments, the amount of detectable compound is determined in a sample at multiple time points, wherein a change in the amount of detectable compound over time indicates that the test compound modulates the activity of the enzyme. In other embodiments, the amount of detectable compound is determined in the sample contacted with a test compound, and compared to the amount of detectable compound determined in a sample containing the enzyme wherein the enzyme has not been contacted with the test compound. In some embodiments, the enzyme may be purified or isolated. In one embodiment, the enzyme may be partially purified. In some embodiments, the test compound may be a small molecule.

In some embodiments, methods for identifying modulators of PGES in which PGES activity is detected, as described in “Methods for Preparing Prostaglandin E Synthase”, U.S. Ser. No. 10/227,617, filed Aug. 23, 2002, may incorporate the activity assays of the present invention.

In one embodiment, assays for identifying such modulators consist essentially in a reaction mixture containing a polypeptide (e.g., an enzyme that uses or produces PGH₂ or analog or fragment thereof optionally fused to a heterologous polypeptide) and a test compound, or a library of test compounds. Such libraries of test compounds, e.g. a plurality of test compounds, may be generated using combinatorial synthetic methods or purchased from vendors.

Any suitable contacting of the compounds can be employed in the methods of the present invention, as one of skill in the art would be able to determine. One exemplary assay of the present invention comprises contacting an enzyme or functional fragment thereof with a test compound or library of test compounds and detecting the formation of complexes. The efficacy of the compound can be assessed by generating dose response curves from data obtained using various concentrations of the test compound. Moreover, a control assay may also be performed to provide a baseline for comparison. In the control assay, enzyme activity is quantitated using the same assay conditions, except in the absence of the test compound.

Assaying biological activity in the presence and absence of a test compound may be accomplished in any vessel suitable for containing the reactants. Such assays may be packaged in kit form. Examples include microtitre plates, test tubes, and micro-centrifuge tubes. In many drug screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they may be generated to permit rapid development and relatively easy detection of an alteration in a molecular target which is mediated by a test compound. Moreover, the effects of cellular toxicity and/or bioavailability of the test compound may be generally ignored in the in vitro system, the assay instead being focused primarily on the effect of the drug on the molecular target as may be manifest in an alteration of binding affinity with other proteins or changes in enzymatic properties of the molecular target. Accordingly, potential modulators may be detected in a cell-free assay generated by constitution of function interactions of interest in a cell lysate. In an alternate format, the assay may be derived as a reconstituted protein mixture which, as described below, offers a number of benefits over lysate-based assays.

In some in vitro embodiments of the present assay, the sample comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-substrate, protein-protein or nucleic acid-protein interaction are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-substrate, protein-protein interaction, or nucleic acid-protein interaction.

F. Kits

The present invention provides kits for use in the practice of the above-described methods. A kit may comprise appropriate reagents for determining the level of enzyme activity, and optionally instructions for their use. A kit may further comprise standards, e.g. an enzyme for which the activity is known. Kit components may be packaged for either manual or partially or wholly automated practice of the foregoing methods. In other embodiments involving kits, this invention provides a kit including compositions of the present invention, and optionally instructions for their use. Such kits may have a variety of uses, including, for example, drug screening.

The invention having been generally described, may be more readily understood by reference to the following examples, which are included merely for purposes of illustration of certain aspects and embodiments of the present invention, and are not intended to limit the invention in any way.

### EXAMPLES

PGES Activity Detection Assays

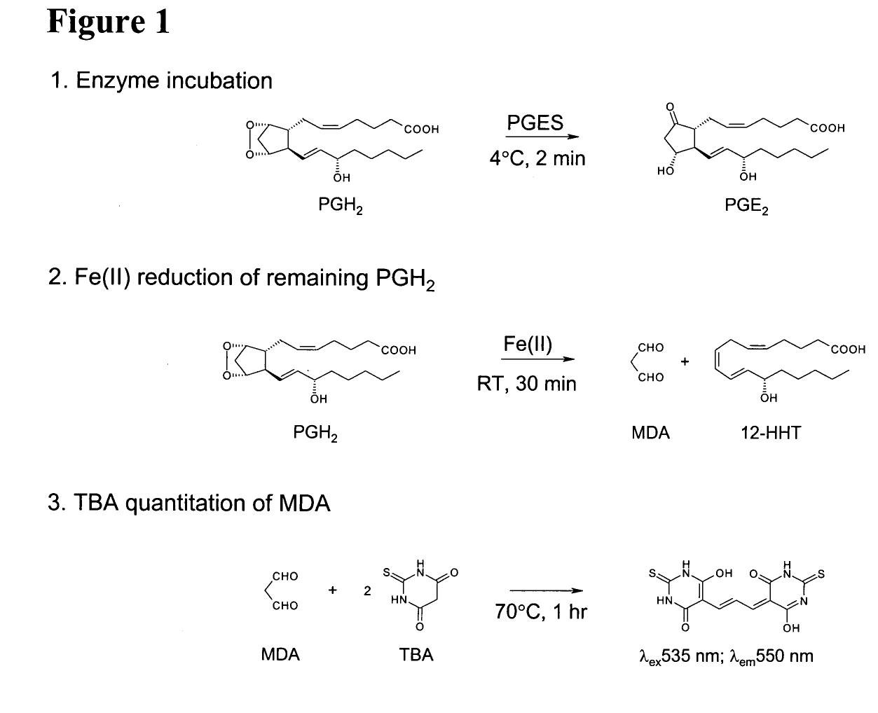

These assays detect the activity of PGES by monitoring its consumption of PGH₂ substrate. In general, PGES and PGH₂ are incubated under conditions which promote their reaction. The incubation is quenched using ferrous chloride (FeCl₂, Sigma F-2130) and citric acid (Sigma C-1909, whereupon remaining substrate is converted to a reactive species and an inert by-product. The reactive species is then reacted with TBA derivatization reagent to form a fluorescent product with excitation max at 530 nm and emission max (em) at 550 nm. FIG. 1 summarizes the chemical reactions occurring in the three main steps of the assays.

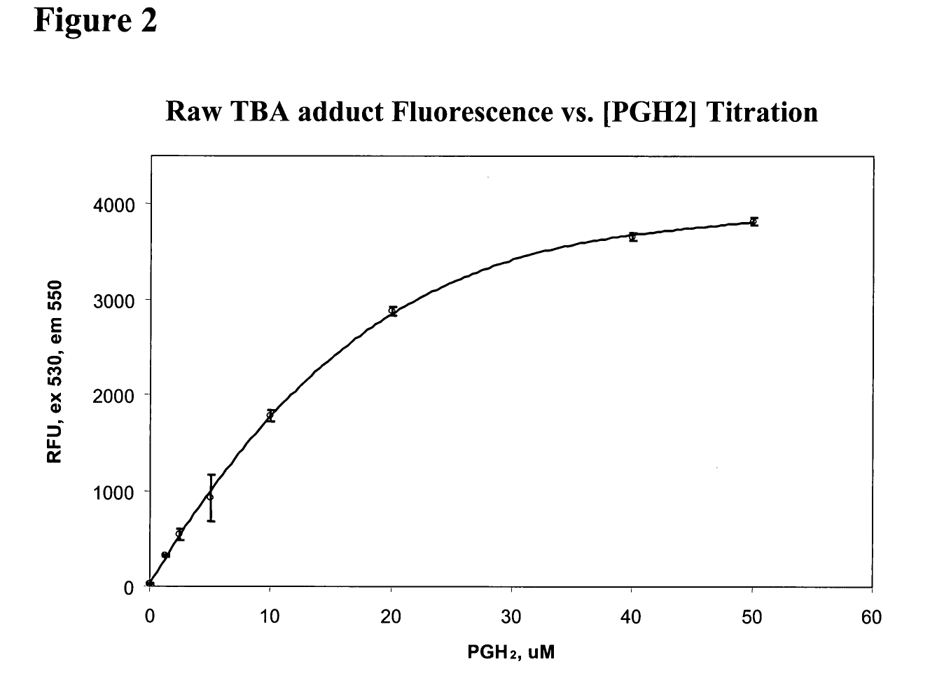

FIG. 2 depicts a titration of PGH₂ using an exemplary embodiment of the assay, wherein the raw fluorescence reading is plotted versus the concentration of PGH₂. Varying concentrations of PGH₂ were incubated with PGES enzyme in 100 uL at 4° C. Incubations were quenched with 50 uL 25 mM FeCl₂, 50 mM citric acid, pH 2.5 at room temperature for 30 min. Thiobarbituric acid (TBA, 0.53%, 150 uL) was added and the plates were developed for 90 min at 70° C. Plates were then cooled to room temperature and read using 530 nm exc/550 nm em on a fluorometer.

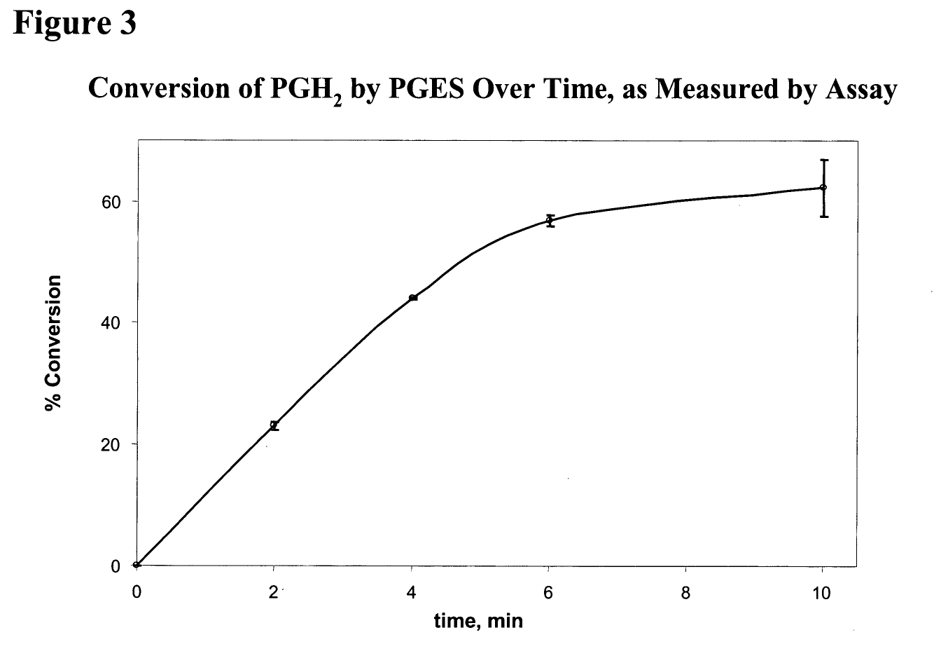

FIG. 3 depicts the percent conversion of PGH₂ by PGES over time, as measured by an exemplary embodiment of the assay. PGES (25 ug/mL) was incubated in 100 uL with 10 uM PGH₂ at 4° C. for varying times. Incubations were quenched with 50 uL 25 mM FeCl₂, 50 mM citric acid, pH 2.5 at room temperature for 30 min. TBA (0.53%, 150 uL) was added and the plates were developed for 90 min at 70° C. Plates were then cooled to RT and read using 530 nm exc/550 nm em. Percent conversion was calculated via the following equation:

% P formed=([P]_{T}/[S]ₒ)×100=(P_{T}/(S_{T}+P_{T}))×100

where:

t=time

0=time zero

P=product

S=substrate

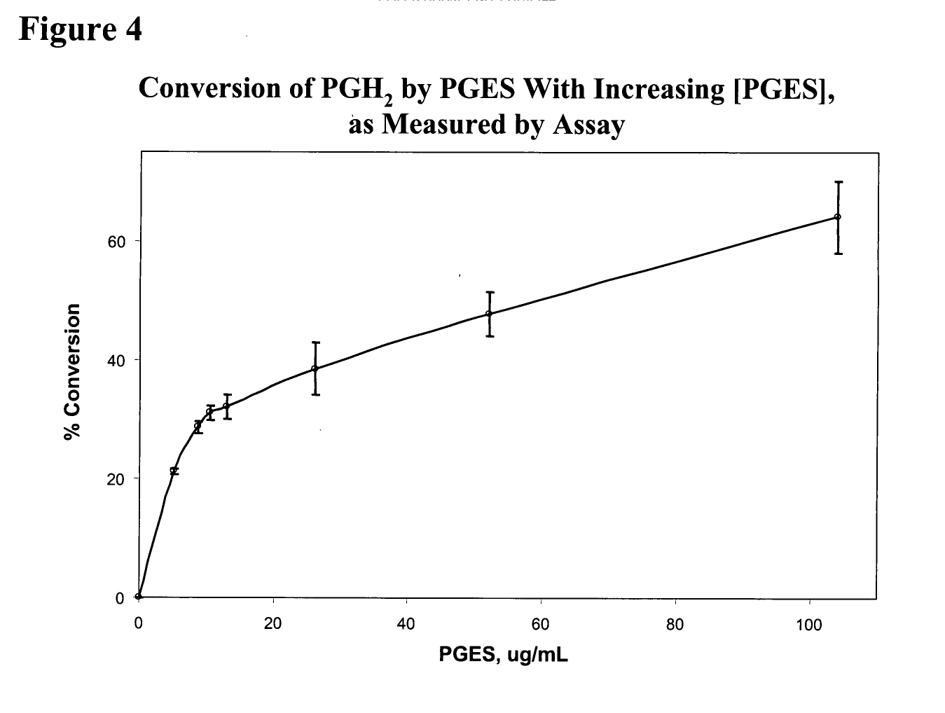

FIG. 4 depicts the percent conversion of PGH₂ by varying PGES concentration, as measured by an exemplary embodiment of the assay. Varying concentrations of PGES were incubated in 100 uL with 10 uM PGH2 at 4° C. for 2 min. Incubations were quenched with 50 uL 25 mM FeCl₂, 50 mM citric acid, pH 2.5 at room temperature for 30 min. TBA (0.53%, 150 uL) was added and the plates were developed for 90 min at 70° C. Plates were then cooled to RT and read using 530 nm exc/550 nm em.

PGES Activity Modulation Assay

This assay may be used to discover and identify compounds which modulate PGES activity. In the following protocol, volumes are per reaction. An assay buffer of 0.01M potassium phosphate pH 7.4 was prepared and used to dilute the PGES enzyme (clone provided by Dr. Per-Johan Jakobsson, Karolinska Institute, Sweden). The PGH₂ substrate (purchased from Dr. Mats Hamburg, Karolinska Institute, Sweden) was diluted in ice cold acetone. 85 ul cold assay buffer were added to each well of a 96-well flat bottom plate using a multidrop pipettor. 5 ul of test compounds (7-fold compression) were added to the plate. Subsequently, 10 ul ice cold enzyme (1:100 diln) solution were added, and the plate transferred into a chemical hood. 5 ul of ice cold PGH₂ substrate (10 uM final) were dispensed into the plate. The plate was incubated 3 minutes at 4° C. Incubations were quenched with 50 uL 25 mM FeCl₂, 50 mM citric acid, pH 2.5 at room temperature for 30 minutes, whereupon remaining substrate is converted to a reactive species and an inert byproduct. This reactive species is then reacted with 150 ul of derivatization solution (TBA, 0.53%) were added, and the plate incubated 90 min at 60-70° C., whereupon a fluorescent product was formed. The plate was read on a Molecular Devices Gemini fluorometer with excitation 530 nm and emission 550 nm.

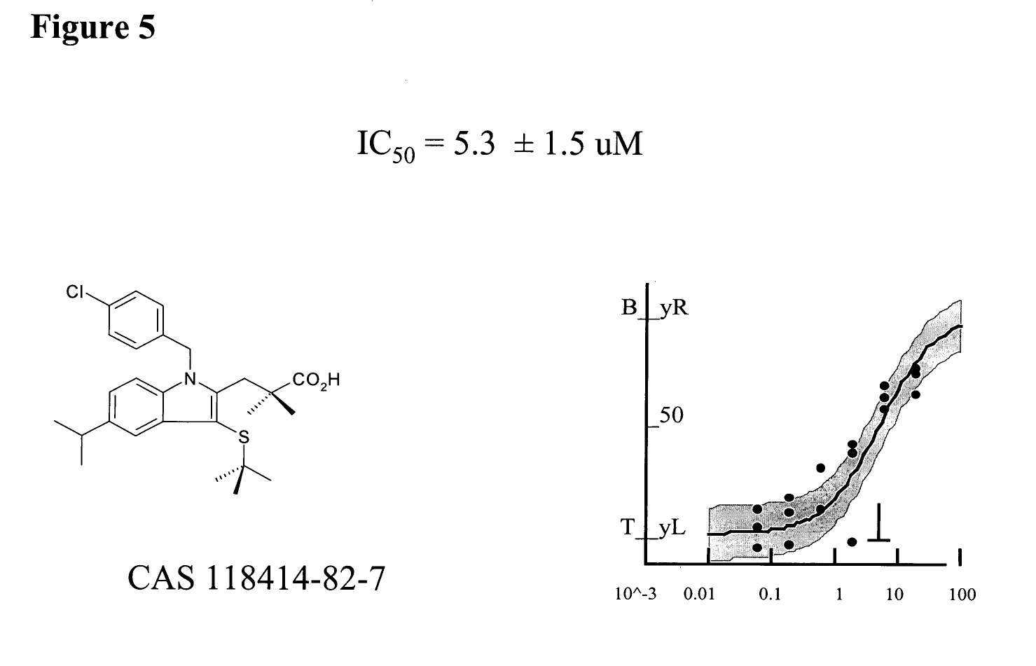

The foregoing assay was used to measure the ability of CAS 118414-82-7 (1H-Indole-2-propanoic acid, 1-[(4-chlorophenyl)methyl]-3-[(1,1-dimethylethyl)thio]-α,α-dimethyl-5-(1-methylethyl)-(9Cl)), a commercially available inhibitor (Cayman Chemical). FIG. 5 depicts the structure of CAS 118414-82-7 and the results of the assay. An IC₅₀ of approximately 5.3 μM was observed, which is consistent with the reported value.

Equivalents

The present invention provides in part novel assays for determining the activity of enzymes that use or produce PGH₂. While specific embodiments of the subject invention have been discussed, the above specification is illustrative and not restrictive. Many variations of the invention will become apparent to those skilled in the art upon review of this specification. The appendant claims are not intended to claim all such embodiments and variations, and the full scope of the invention should be determined by reference to the claims, along with their full scope of equivalents, and the specification, along with such variations.

All publications and patents mentioned herein, including those items listed below, are hereby incorporated by reference in their entireties as if each individual publication or patent was specifically and individually indicated to be incorporated by reference. In case of conflict, the present application, including any definitions herein, will control.

Additional References

Janero, D. R., (1990) Free Radical Biol. Med. 9:515-540; Ledergerber, D. and Hartmann, R. W. (1995) J. Enzyme Inhib. 9:253-261; Hamberg, M. and Samuelson, B. (1974) Proc. Natl. Acad. U.SA. 71:3400-3404; Konturek, S. J., and Pawlik, W. (1986) Digestive Diseases and Sciences 31:65-195; and Hammarstrom, S. (1982) Arch. Biochem. Biophys. 214:431-445.

I claim:

1. A method for determining the activity of an enzyme that uses PGH₂ but does not produce malondialdehyde, comprising:
(a) contacting a sample with a reducing agent under conditions suitable to substantially convert PGH₂ into malondialdehyde and thereby obtain a reacted sample;
(b) contacting the reacted sample with a malondialdehyde detection reagent under conditions suitable to substantially convert the malondialdehyde into a detectable compound; and
(c) determining the amount of the detectable compound, wherein the activity of the enzyme in the sample is inversely proportional to the amount of the detectable compound.

2. The method of claim 1, wherein the enzyme is selected from the group consisting of prostaglandin synthases and prostacyclin synthases.

3. The method of claim 2, wherein the enzyme is a prostaglandin synthase.

4. The method of claim 3, wherein the enzyme is prostaglandin E synthase (PGES).

5. The method of claim 1, wherein the reducing agent is ferrous chloride.

6. The method of claim 1, wherein the detection reagent is 2-thiobarbituric acid (TBA).

7. The method of claim 1, wherein the detection reagent is a 2-thiobarbituric acid derivative.

8. The method of claim 1, wherein the detectable compound is a fluorescent compound.

9. A method for determining the activity of an enzyme that produces PGH₂ but does not produce malondialdehyde comprising:
(a) contacting a sample with a reducing agent under conditions suitable to substantially convert PGH₂ into malondialdehyde and thereby obtain a reacted sample;
(b) contacting the reacted sample with a malondialdehyde detection reagent under conditions suitable to substantially convert the malondialdehyde into a detectable compound; and
(c) determining the amount of the detectable compound, wherein the activity of the enzyme in the sample is proportional to the amount of the detectable compound.

10. The method of claim 9, wherein said enzyme is a PGH2 synthase.

11. The method of claim 9, wherein the reducing agent is ferrous chloride.

12. The method of claim 9, wherein the detection reagent is 2-thiobarbituric acid (TBA).

13. The method of claim 9, wherein the detection reagent is a 2-thiobarbituric acid derivative.

14. The method of claim 9, wherein the detectable compound is a fluorescent compound.

15. A method of identifying a modulator of an enzyme that uses or produces PGH₂ but does not produce malondialdehyde comprising:
(a) contacting a sample containing an enzyme with a test compound;
(b) contacting the sample with a reducing agent under conditions appropriate to convert PGH₂ into malondialdehyde and thereby obtain a reacted sample;
(c) contacting the reacted sample with a malondialdehyde detection reagent under conditions appropriate to convert the malondialdehyde into a detectable compound; and
(d) determining the amount of the detectable compound, wherein the amount of detectable compound is used to determine whether or not the test compound modulates the activity of the enzyme.

16. The method of claim 15, wherein said enzyme is isolated or purified.

17. The method of claim 15, wherein said enzyme is partially purified.

18. The method of claim 15, wherein said test compound is a small molecule.

19. The method of claim 15, wherein the amount of detectable compound is determined in a sample at multiple time points, and wherein a change in the amount of detectable compound over time indicates that the test compound modulates the activity of the enzyme.

20. The method of claim 15, wherein the amount of detectable compound is determined in the sample contacted with a test compound, and compared to the amount of detectable compound determined in a control sample containing the enzyme wherein the enzyme has not been contacted with the test compound.

21. A kit for determining the activity of an enzyme that uses or produces PGH2 but does not produce malondialdehyde as a product comprising a reducing agent and a malondialdehyde detection reagent.

22. The kit of claim 21, further comprising instructions.
